(19) United States
(12) Patent Application Publication
(10) Pub. No.: US 2005/0186168 A1
(43) Pub. Date: Aug. 25, 2005

(54) Skin remodeling and regenerative compositions containing elastin peptide ligands having the amino acid sequence (XGXXPG)n

(76) Inventor: Eric L. Albin, Aventura, FL (US)

Correspondence Address: Eric L Albin, 3733 NE 208 Terrace, Aventura, FL, 33180, US

(21) Appl. No.: 10/785,173

(22) Filed: Feb. 24, 2004

(51) Int. Cl. A61K 38/10; A61K 38/08; A61K 7/11; A61K 7/06

(52) U.S. Cl. 424/70.14; 514/12; 514/14; 514/17; 514/23

(57) ABSTRACT

The present invention relates to skin care compositions providing a method of enhancing the remodeling of age damaged and sun damaged skin in mammals utilizing an elastin peptide (EP) ligand of the general formula (GXXPG)n and in particular the sequences of (VGVAPG)n and/or (GVAPGV)n, and/or (VAPGVG)n and/or (APGVGV)n and/or (PGVGVA)n and/or (GVGVAP)n and a galactosugar which modulates the effect of the elastin peptide ligand(EP) on its lectin the Elastin-Binding Receptor (EBR). wherin A is a peptide-forming residue of L-alanine; G is a peptide-forming residue of glycine P is a peptide-forming residue of L-proline V is a peptide-forming residue of L-valine X is a peptide-forming residue of a single unspecified amino acid
Peptide representations in this application conform to the standard practice of writing the NH₂-terminal amino acid residue at the left of the formula and the CO₂ H-terminal amino acid residue at the right.

### REFERENCES CITED

### U.S. PATENT DOCUMENTS

U.S. PTO Application 03970 U.S. PTO Ser. No. 10/657,924 filed Sep. 10, 2003

This patent application differs U.S. PTO Ser. No. 10/657,924 in that it does not include copper-peptide complexes in the formulations and galactosugar bearing moieties may or may not be added to modulate the stimulating effects of the elastin peptide on metallolprotinase and collagenolytic enzymes.

### OTHER PUBLICATIONS

1. Hayflick L, Moorhead P: The serial cultivation of human diploid cell strains. Expl Cell Res 25:585-621, 1961.
2. Macieira-Coelho A: Cancer and aging. Exper Gerontol 21:483-495, 1986.
3. Brenner S. ed., Telomeres and telomerase, 1997, Ciba Foundation Symposium 211, John Wiley & Sons, Chichester.
4. Gown A M, Tsukada T, Ross R: Human atherosclerosis II. Immunocytochemical analysis of the cellular composition of human atherosclerotic lesions. Am J Pathol 125:191-207, 1986.
5. Raines E W, Ross R: Mechanisms of plaque formation: cellular changes and the possible role of growth-regulatory molecules. In: Atherosclerosis Rev.(Eds: Weber P C, Leaf A). Raven Press, New York, 1991, 23:143-152.
6. Robert L: Les horloges biologiques. 1989, Flammarion, Paris.
7. Verzar F: Aging of the collagen fiber. In: International review of connective tissue research. (Ed: Hall DA). Academic Press, New York, 1964, 2:243-300.
8. Ikan R., Ed. The Maillard reaction. Consequences for the chemical and life sciences. John Wiley & Sons, Chichester, 1996.
9. Fodil-Bourahla I, Drubaix I, Robert L: Effect of in vitro aging on the biosynthesis of glycosaminoglycans by human skin fibroblasts. Modulation by the elastin-laminin receptor. Mechanisms of Ageing and Development 106:241-260, 1999.
10. Labat-Robert J, Kern P, Robert L: Biomarkers of connective tissue aging: biosynthesis of fibronectin, collagen type III and elastase. Ann New York Acad Sci 673:16-22, 1992.
11. Labat-Robert J, Potazman J P, Derouette J C, Robert L: Age-dependent increase of human plasma fibronectin. Cell Biol Int 5:969-973, 1981.
12. Robert L, Robert A M: Elastin, elastase and arteriosclerosis. In: “Frontiers of Matrix Biologie”. (Eds: Robert A M, Robert L). Karger, Basel, 1980, 8:130-173
13. Robert L: Aging of the vascular-wall and atherosclerosis. Experimental Gerontology 34:491-501, 1999.
14. Robert L, Robert A M, Jacotot B: Elastin-elastase atherosclerosis revisited. Atherosclerosis 140:281-295, 1998.
15 Urry D W:, Sequential polypeptides of elastin: structural proper-ties and molecular pathologies. In: Frontiers of Matrix Biology
16. Kielty, C. M., Woolley, D. E., Whittaker, S. P. and Shuttleworth, C. A. (1994). Catabolism of intact fibrillin microfibrils by neutrophil elastase, chymotrypsin and trypsin. FEBS Lett. 351, 85-89.
17. Ashworth, J. L., Sherratt, M. J., Rock, M. J., Murphy, G., Shapiro, S. D., Shuttleworth, C. A. and Kielty, C. M. (1999c). Fibrillin turnover by metalloproteinases: implications for connective tissue remodelling. Biochem. J. 340,171-181.
18. Watson, R. E. B., Griffiths, C. E. M., Craven, N. M., Shuttleworth, C. A. and Kielty, C. M. (1999). Fibrillin-rich microfibrils are reduced in photoaged skin. Distribution at the dermal-epidermal junction. J. Invest. Dermatol. 112,782-787.
19. Mecham, R. P. and Davis, E. C. (1994). Elastic fiber structure and assembly. In Extracellular Matrix Assembly and Structure (ed. P. D. Yurchenco, D. E. Birk and R. P. Mecham), pp. 281-314. New York: Academic Press.
20. Bellingham, C. M., Woodhouse, K. A., Robson, P., Rothstein, S. J. and Keeley, F. W. (2001). Self-aggregation of recombinantly expressed human elastin polypeptides. Biochim. Biophys. Acta 1550, 6-19.
21. Toonkool, P., Jensen, S. A., Maxwell, A. L. and Weiss, A. S. (2001). Hydrophobic domains of human tropoelastin interact in a context-dependent manner. J. Biol. Chem. 275, 44575-44580.
22. Ashworth, J. L., Kelly, V., Wilson, R., Shuttleworth, C. A. and Kielty, C. M. (1999a). Fibrillin assembly: dimer formation mediated by amino-terminal sequences. J. Cell Sci. 112, 3549-3558.
23. Trask, T. M., Ritty, T. M., Broekelmann, T., Tisdale, C. and Mecham, R. P. (1999). N-terminal domains of fibrillin 1 and fibrillin 2 direct the formation of homodimers: a possible first step in microfibril assembly. Biochem. J. 340, 693-701.
24. Sechler, J. L., Rao, H., Cumiskey, A. M., Vega-Colón, I., Smith, M. S., Murata, T. and Schwarzbauer, J. E. (2001). A novel fibronectin binding site required for fibronectin fibril growth during matrix assembly. J. Cell Biol. 154, 1081-1088.
25. Pereira, L., D'Alessio, M., Ramirez, F., Lynch, J. R., Sykes, B., Pangilinan, T. and Bonadio, J. (1993). Genomic organization of the sequence coding for fibrillin, the defective gene product in Marfan syndrome. Hum. Mol. Genet. 2, 961-968.
26. Zhang, H., Apfelroth, S. D., Hu, W., Davis, E. C., Sanguineti, C., Bonadio, J., Mecham, R. P. and Ramirez, F. (1994). Structure and expression of fibrillin-2, a novel microfibrillar component preferentially located in elastic matrices. J. Cell Biol. 124, 855-863.
27. Nagase, T., Nakayama, M., Nakajima, D., Kikuno, R. and Ohara, O. (2001). Prediction of the coding sequences of unidentified human genes. XX. The complete sequences of 100 new cDNA clones from brain with code for large proteins in vitro. DNA Res. 8, 85-95.
28. Baccarani-Contri, M., Vincenzi, D., Cicchetti, F., Mori, G. and Pasquali-Ronchetti, I. (1990). Immunocytochemical localization of proteoglycans within normal elastic fibres. Eur. J. Cell Biol. 53, 305-312.
29. Tiedemann, K., Batge, B., Müller, P. K. and Reinhardt, D. P. (2001). Interactions of fibrillin-1 with heparin/heparan sulphate, implications for microfibrillar assembly. J. Biol. Chem. 276, 36035-36042.
30. Brown-Augsburger, P., Tisdale, C., Broekelmann, T., Sloan, C. and Mecham, R. P. (1995). Identification of an elastin crosslinking domain that joins three peptide chains. J. Biol. Chem. 270, 17778-17783.
31. 1. Hayflick L, Moorhead P S: The serial cultivation of human diploid cell strains. Expl Cell Res 25:585-621, 1961.
32. Toonkool, P., Jensen, S. A., Maxwell, A. L. and Weiss, A. S. (2001). Hydrophobic domains of human tropoelastin interact in a context-dependent manner. J. Biol. Chem. 275, 44575-44580.
33. Csiszar, K. (2001). Lysyl oxidases: a novel multifunctional amine oxidase family. Prog. Nucleic Acid Res. Mol. Biol. 70, 1-32.
34. Borel, A., Eichenberger, D., Farjanel, J., Kessler, E., Gleyzal, C., Hulmes, D. J. S., Sommer, P. and Font, B. (2001). Lysyl oxidase-like protein from bovine aorta. J. Biol. Chem. 276, 48944-48949.
35. Boudier, C., Godeau, G., Hornebeck, W., Robert, L., and Bieth, J. G. (1991) Am. J. Respir. Cell Mol. Biol. 4, 497-503
36. Hinek, A., Rabinovitch, M., Keeley, F., Okamura-Oho, Y., and Callahan, J. (1993) J. Clin. Invest. 91, 1198-1205
37. Senior, R. M., Griffin, G. L., Mecham, R. P., Wrenn, D. S., Prasad, K. U., and Urry, D. W. (1984) J. Cell Biol. 99, 870-874
38. Long, M. M., King, V. J., Prasad, K. U., and Urry, D. W. (1983) Biochim. Biophys. Acta 968, 300-311
39. Yusa, T., Blood, C. H., and Zetter, B. R. (1989) Am. Rev. Respir. Dis. 140, 1458-
40. Grosso, L. E., and Scott, M. (1993) Matrix 13, 157-164
41. Hauck, M., Seres, I., Kiss, I., Saulnier, J., Mohacsi, A., Wallach, J., and Fülöp, T., Jr. (1995) Biochem. Mol. Biol. Int. 37, 45-55
42. Uemura, Y., and Okamoto, K. (1997) Biochem. Mol. Biol. Int. 41, 57-64
43. Castiglione-Morelli, M. A., Bisaccia, F., Spisani, S., De Biasi, M., Traniello, S., and Tamburro, A. M. (1997) J. Pept. Res. 49, 492-499
44. Ghuysen-Itard, A. F., Robert, L., and Jacob, M. P. (1992) C. R. Acad. Sci. III (Paris) 315, 473-478
45. Wachi, H., Seyama, Y., Yamashita, S., Suganami, H., Uemura, Y., Okamoto, K., Yamada, H., and Tajima, S. (1995) FEBS Lett. 368, 215-219
46. Kamoun, A., Landeau, J. M., Godeau, G., Wallach, J., Duchesnay, A., Pellat, B., and Hornebeck, W. (1995) Cell Adhes. Commun. 3, 273-281
47. Jung, S., Rutka, J. T., and Hinek, A. (1998) J. Neuropathol. Exp. Neurol. 57, 439-448
48. Jacob, M. P., Fülöp, T., Jr., Foris, G., and Robert, L. (1987) Proc. Natl. Acad. Sci. U.S.A. 84, 995-999
49. Varga, Z., Jacob, M. P., Robert, L., and Fülöp, T., Jr. (1989) FEBS Lett. 258, 5-8
50. Faury, G., Ristori, M. T., Verdetti, J., Jacob, M. P., and Robert, L. (1995) J. Vasc. Res. 32, 112-119
51. Faury, G., Garnier, S., Weiss, A. S., Wallach, J., Fülöp, T., Jr., Jacob, M. P., Mecham, R. P., Robert, L., and Verdetti, J. (1998) Circ. Res. 82, 328-336
52. Faury, G., Usson, Y., Robert-Nicoud, M., Robert, L., and Verdetti, J. (1998) Proc. Natl. Acad. Sci. U.S.A. 95, 2967-2972
53. Lograno, M. D., Bisaccia, F., Ostuni, A., Daniele, E., and Tamburro, A. M. (1998) Int. J. Biochem. Cell Biol. 30, 497-503
54. Fülöp, T., Jr., Jacob, M. P., Varga, Z., Foris, G., Leovey, A., and Robert, L. (1986) Biochem. Biophys. Res. Commun. 141, 92-98
55. Brassart, B., Randoux, A., Hornebeck, W., and Emonard, H. (1998) Clin. Exp. Metastasis 16, 489-500
56. Fodil-Bourahla, I., Drubaix, I., Robert, L., Labat-Robert, J. (1999) Gerontology January-February; 45(1):23-30
57. Brassart, B., Fuchs, P., Huet, E., Alix, A. J. P., Wallach, J., Tamburro, A. M., Delacoux, F., Haye, B., Herve. E., Hornbeck, W., Debelle. L. (2000) JBC. 276, 5222-5227
58. Mecham, R. P., Hinek, A., Griffin, G. L., Senior, R. M., and Liotta, L. A. (1989) J. Biol. Chem. 264, 16652-16657
59. Indik, Z., Yeh, H., Ornstein-Goldstein, N., Sheppard, P., Anderson, N., Rosenbloom, J. C., Peltonen, L., and Rosenbloom, J. (1987) Proc. Natl. Acad. Sci. U.S.A. 84, 5680-5684
60. Yeh, H., Ornstein-Goldstein, N., Indik, Z., Sheppard, P., Anderson, N., Rosenbloom, J. C., Cicila, G., Yoon, K., and Rosenbloom, J. (1987) Collagen Relat. Res. 7, 235-247
61. Fülöp, T., Jr., Wei, S. M., Robert, L., and Jacob, M. P. (1990) Clin. Physiol. Biochem. 8, 273-282
62. Hornebeck, W., Gogly, B., Godeau, G., Emonard, H., and Pellat. B. (1999) Ann. N.Y. Acad. Sci. 878, 625-628. Hornebeck, W., Gogly, B., Godeau, G., Emonard, H., and Pellat, B. (1999) Ann. N.Y. Acad. Sci. 878, 625-628
63. Hinek, A. (1996) Biol. Chem. 377, 471-480
64. Wrenn, D. S., Griffin, G. L., Senior, R. M., and Mecham, R. P. (1986) Biochemistry 25, 5172-5176
65. Peterszegi, G., Texier, S., Robert, L. (1999) Eur J Clin Invest. 29(2):166-72
66. Maquart, Pickart, Laurent, Gillery, Monboisse, Borel, FEBS Lett. 238, 1988, 343;
67. Oddos, Jumeau-Lafond, Ries, Amer. Acad. Derm. Abstract P72, February 2002;
68. Wegrowski, Maquart, Borel, Life Sci. 51, 1992, 1049;
69. Simeon, Wegrowski, Bontemps, Maquart, J. Invest. Dermatol., 115, 2000, 962;
70. Maquart, Bellon, Chaqour, Wegrowski, Monboisse, Chastang, Birembaut, Gillery, J. Clin. Invest., 92, 1993, 2368;
71. Simeon, Monier, Emonard, Gillery; Birembaut, Hornebeck, Maquart, J. Invest. Dermatol., 112, 1999, 957
72. Simeon, Emonard, Hornebeck, Maquart, Life Sci., 67, 2000, 2257
73. Maquart, Simeon, Pasco, Monboisse, J. Soc. Biol., 193, 1999, 423; McCormack, Nowak, Koch, Arch. Facial Plast. Surg., 3, 2001, 28 1997).
74. Jacob, M. P., and Hornebeck, W. (1985) Front. Matrix Biol. 10, 92-129

### BACKGROUND OF THE INVENTION

Cellular Aging

Hayflick and Moorehead(1) reported that cells in culture, kept in standardized conditions, can undergo only a limited number of population doublings. A negative correlation was found between the maximal number of population doublings and the age of the donor of skin fibroblasts (see 2 for a review). This is attributed to the shortening of telomeres at every cellular division.(3) Telomerase, the enzyme capable of resynthesising the telomeres on each end of chromosomes is repressed in most somatic cells but re-expressed in most malignant cells.

Cells at the end of their replicative life span do not die. Their morphologic changes, enlarged cell size, vacuolization, loss of mitochondrial integrity etc. are well characterized. (2) These cells are unable to divide but could be kept for long periods in culture.(4,5)

Most cells of the organism do not exhaust their division capacity during maximal human life expectancy 100 years±20). The keratinocytes of the epidermis, the epithelial cells of the mucosal layers of the gastrointestinal tract and the bone marrow cells, are in constant mitotic activity, all other cell-types of the organism do not divide regularly after they reach their mature phenotype. Stem cells can be demonstrated in several tissues retain the possibility of further divisions and differentiation. Cellular aging reflects the loss of proliferation and cellular function. (for a review see 6).

Aging of the Extracellular Matrix

Postsynthetic molecular aging was first described by Verzar (7) on the rat tail tendon. He observed the mechanical force of thermal retraction of collagen fibers increased exponential with age This relationship between resistance to heat denaturation and age of collagen fibers was verified in all species studied. Verzar attributed this to an age-dependent increase of cross-linking of collagen. The crosslinks were not the Schiff base- or aldol-condensation products of lysine or hydroxylysines which are the “normal” cross-links. Age-dependent increase of collagen cross-links is the result of the Maillard reaction, the progressive non enzymatic glycanation of amino groups on proteins, followed by the formation of polycyclic, aromatic compounds designated as “advanced glycosylation end products” or AGE. (for a review see 8),

Some AGE-products can release free radicals. The speed of glycanation increases with the concentration of reactants and thus advances much faster in hyperglycemic diabetics. These Glycanated proteins have altered biological properties such as loss of elasticity of elastin. Other matrix macromolecules, proteoglycans and structural glycoproteins also exhibit age-dependent variations. The rate of biosynthesis of matrix macromolecules changes with age. Some decline, others increase. The biosynthesis of hyaluronan, a glycosaminoglycan, was shown to decrease with aging of human skin fibroblasts.(9) Post synthetic modifications of proteoglycans and glycosaminoglycans consist of both enzymatic and non enzymatic (free radical-mediated) degradation. Fibronectin, the most abundant structural glycoprotein, is increasingly synthesized by in vivo and in vitro with cell aging.(10,11) Fibronectin proteolytic degradation fragments are also increasing with age.

Free radical generation resulting from metabolic processes also contributes to matrix degeneration. Free radical scavenging systems are present in cells and tissues, their efficiency is far from 100% and decrease with age.

Elastin fibers fixate calcium and lipids, loose elasticity and are degraded by elastase-type endopeptidases. (12,13,14,15)

Elastic Fibers

Elastic fibers are essential insoluble extracellular matrix (ECM) macromolecules comprising an elastin core surrounded by a framework of fibrillin-rich microfibrils. It is these fibers that endow connective tissues with resilience. The elastic fiber can stretch more than twice its resting length and passively recoil. This elasticity is critical to the function of arteries, lungs, skin and all other dynamic connective tissues. Elastic fibers complement collagen fibers, which impart tensile strength.

While elastin is insoluble, ECM enzymes such as metalloproteinases and serine proteases are able to cleave elastic fiber. (16,17). Elastin may also lose some of its elastic properties by non-enzymatic glylcanation and oxidative reactions. Loss of elasticity is a major contributing factor in the ageing of connective tissues and age related skin damage. (18).

In the skin dermal fibroblasts generate fibrillin, which is assembled into microfibrils. While not fully elucidated current knowledge indicates that the microfibrils are assembled in the ECM next to the fibroblast cellular membrane. The microfibril serves as a structural framework for the deposition of tropoelastin (the soluble precursor of elastin) (19). In the arterial system fibrillin and elastin are produced by smooth muscle cells instead of fibroblasts.

Tropoelastin undergoes a process called coascervation. Coascervation is the self-induced ability to organize into a polymeric structure that is dictated by its chemical geometry. (20,21). Tropoelastin binds to the microfibril, and then coascervates. The final step is the internal cross-linking catalyzed by the copper containing enzyme lysyl oxidase and is inhibited by the lack of copper (19).

Microfibril assembly is a cell-regulated process that is independent of tropoelastin synthesis. (22,23).

Elastic fibers are a composite of an outer microfibrillar framework and an inner core of cross-linked elastin. The architecture of mature elastic fibers is highly tissue specific and relates to the functional demands of the tissue. The reticular dermis of skin contains thick, horizontally arranged elastic fibers, the papillary dermis contains thinner perpendicular elastic fibers that interlace into the dermal-epidermal junction.

Microfibrillar molecules. It has been recognized for many years that microfibrils form a template for elastin. Microfibrils assemble close to the cell surface in a process that might require receptors, as shown for fibronectin, in which dimer interactions with 5β1 integrins induce a conformation change that leads to linear assembly (24).

Fibrillins are the principal structural components of elastic-fiber-associated microfibrils. Fibrillin-1 and fibrillin-2 are encoded by genes on chromosomes 15 and 5, respectively (25,26), and a third, closely related, fibrillin-3 gene has been identified on chromosome 19 (27).

It is not known if fibrillin can self assemble. However, microscopy studies indicate that assembly occurs in association with the mesenchymal cell surface and suggests cell surface receptor functions.

Different extracellular microfibril populations have been identified. The extracellular environment plays a major role in regulating microfibril fate.

Several proteoglycans (PGs) also engage in critically important interactions with microfibrils and contribute to their integration into the surrounding extracellular matrix (ECM). (28) Binding of fibrillin-1 to heparan sulfate chains is a prerequisite for assembly. It is not clear at this stage whether proteoglycans secreted into the extracellular space or cell membrane-associated proteoglycans are necessary for microfibrillar assembly. Since fibrillin-1 only binds to highly sulfated and iduronated regions within a glycosaminoglycan chain, the patterns of high and low sulfated regions could determine a spatial arrangement of fibrillin-1 necessary to facilitate fibrillin-1 self-interactions or for disulfide bond formation, which is known as one of the initial steps in fibrillin-1 assembly (13). (iii) Binding of fibrillin-1 to glycosaminoglycans potentially confers conformational changes to the fibrillin-1 protein necessary to expose epitopes for assembly. Relatively fast on and off rates for protein binding to heparan sulfate chains are ideal to support the proposed functions. For example the glycosaminoglycan chains could provide surfaces upon which fibrillin-1 molecules quickly find each other in order to concentrate, to align in the proper register, and to change its conformation. Once the supported step in the assembly process has been “catalyzed,” the fibrillin-1 molecules or multimers could be released immediately into the extracellular matrix. (29)

Tropoelastin synthesized by ribosomes of the rough endoplasmic reticulum and processed by the Golgi apparatus has a molecular mass of 70 kDa and alternating hydrophobic and crosslinking domains (19,30). Interactions between hydrophobic domains are important in self-assembly coascervation and essential for elasticity (Bellingham et al., 2001; Toonkool et al., 2001). Lysyl-oxidase in the presence of copper catalyzes the cross linking of lysine molecules thus forming covalent lysyl-derived insoluble elastin (Csiszar, 2001), (Borel et al., 2001).

The extracellular matrix protein elastin is responsible for the elastic properties of tissues such as lung, skin, and large arteries. Due to its numerous cross-links and the extreme hydrophobicity of its tropoelastin chains, elastin is highly resistant to proteolysis. However, during inflammatory disorders, proteinases secreted from polymorphonuclear neutrophils, such as elastase, cathepsin G, and gelatinase B may cause significant elastolysis (35).

Stimulation of Elastin and Collagen Production Elastin Peptides(EP) and the Elastin-Binding Protein (EBP)

The 67-kDa Elastin Binding Protein (EBP) Mediates the Effect of Elastin Peptides on Fibroblasts, smooth muscle cells, monocytes and lymphcytes. It has been established that peptides derived from elastin or from the hydrophobic domains of tropoelastin interact with cells via a cell surface-resided 67-kDa elastin-binding protein (36). The binding of elastin peptides to the elastin-binding protein (EBP) has been shown to be responsible for chemotaxis, the migration of fibroblasts and monocytes to the complex site (37-43), stimulation of cell proliferation (44-47) ions flux modifications (48,49), vasorelaxation (50-53), enzyme secretion (54,55) and stimulation of the production of proteins, elastin, collagen and fibronectin(56), and stimulation of the production of glycosaminoglycans (GAG) and hyaluronin.(9).

VGAPG elastin fragment a major ligand of the elastin receptor resulted in stimulation of pro-MatrixMetalloProteinase-1 (MMP-1) and was correlated with enhanced expression of MMP-1 mRNA levels, suggesting that elastin peptides up-regulated MMP-1 at the expression level. Standardization of data using a 36B4 cDNA probe demonstrated that, after 24 h of culture, MMP-1 mRNA levels were increased 8-fold. (57)

Elastin, elastolysate, tropoelastin, and insoluble bovine elastin (kE) concentrations as low as 50 μg/ml proved sufficient to stimulate pro-MMP-1 production, comparable stimulation levels could only be reached with 200 μg/ml VGVAPG, which is a major ligand domain of the elastin receptor. This concentration was 3-4 orders of magnitude higher than the one required for some other elastin peptide-induced activities such as chemotaxis and the production of GAG and hyaluronin. (57) Thus the chemotaxis, protein and elastin producing responses induced by VGVAPG may be induced while the pro-MatrixMetalloProteinase production enhancing functions are not induced by utilizing concentrations of VGAPG of >50-<200 ug/ml.

Elastin peptides bearing the VGVAPG sequence have been shown as a principal ligand of the 67-kDa EBP. (58) It has also been established that the EBP interaction with this elastin-derived domain was only possible in the absence of galactosugars, (i.e. melibiose, lactose) which otherwise may bind to a separate galactolectin binding domain of the EBP and make this molecule unreceptive for elastin peptides. Thus, the addition of such galactosugar-bearing moieties as lactose or melibiose blocks or modulates the specific interaction between elastin peptides and the EBP. (36)

The addition of 1 mM lactose to the fibroblast culture medium resulted in a substantial (35%) inhibition of kE-stimulatedpro-MMP-1 production. In the same conditions, VGVAPG-stimulating effect was inhibited by 80% .(57) These data strongly suggested that binding of VGVAPG on the 67-kDa EBP could explain pro-MMP-1 up-regulation. It needs to be emphasized, however, that stimulation of pro-MMP-1 by interleukin-1 could not be blocked by lactose, and lactose alone had no effect on pro-MMP-1 accumulation stimulated by interleukin-1. (57)

These results further implicate involvement of the EBP in the signaling pathways leading to up-regulation of MMP-1 and are consistent with the EBP-dependent signaling during elastin peptide-stimulated chemotaxis of leukocytes (48). It must also be stressed that elastin peptide-dependent induction of pro-MMP-1 could not be blocked by an interleukin-1 receptor antagonist.(57)

Peptides Containing the GXXPG Consensus Sequence Up-regulate Pro-MMP-1—The multiple hydrophobic VGVAPG sequences occur exclusively in tropoelastin region encoded by exon 24 (59). In bovine tropoelastin, it repeats twice, and in human tropoelastin, it repeats six times (60). The synthetic peptide sequence VGVAPG is highly effective in stimulation of pro-MMP-1 production. Other domains bearing a similar conformation were tested to determine if they could evoke similar cellular effects.

Only peptides bearing the GXXPG sequence could induce pro-MMP-1, suggesting that this sequence was necessary for correct binding to the EBP. VGVAPG induced pro-MMP-1 to a substantial level (1 ng/h/10⁵ fibroblasts), and GVAPGV was 20% more efficient. (57)

Western blotting indicated that fibroblasts stimulated either with kE or with VGVAPG significantly up-regulated expression of proteins reacting with anti-MMP-3 antibody. Two immuno-reactive bands corresponding to pro-MMP-3 glycosylated (60 kDa) and nonglycosylated (57 kDa) isoforms were observed and the accumulation of pro-MMP-3 in the medium was decreased when cells were treated with 1 mM lactose. Furthermore the appearance of these pro-MMP-3 bands was consistent with an increased MMP-3 gene expression in kE-stimulated fibroblasts. (57)

In cultures of unstimulated fibroblasts, the basic level of detected MMPs was not sufficient to up-regulate a basic level of collagenolysis. In kE-stimulated fibroblasts, the addition of plasmin triggered a massive activation of pro-MMP-1 to a collagenolytic enzyme.(57)

Interaction of elastin-derived peptides with the cell surface EBP leads to up-regulation of diverse gene expression and multiple cellular effects (48,51). Both tropoelastin and elastin degradation products are potent inducers of collagenolytic enzyme expression in human skin fibroblasts. An effect was attained even at elastin-derived peptide concentrations close to those determined in physiological fluids (61) and enhanced at higher concentrations (62). This effect could be largely inhibited in the presence of lactose and reproduced by stimulation with VGVAPG and other peptides bearing the GXPG consensus sequence suggests involvement of the EBP in signaling triggering pro-MMP-1 and pro-MMP-3 up-regulation.(57)

The assembly of tropoelastin into mature elastic fibers is also directed by the EBP (63). The VGVAPG cell recognition domains are accessible on the surface of growing elastic fibers as shown using specific monoclonal antibodies (64). However, these sequences are probably masked by the fibrillin template. Leukocytes can release potent elastolytic enzymes which would unmask these domains allowing them to bind to the EBP.

Matrix proteins like elastin, laminins, collagens, fibrillins, or fibronectin contain several GXXPG consensus sequences. Stimulation of MMP-1 expression could also be achieved using the laminin-derived LGTIPG peptide. Peptides bearing GPG conformation, regardless of origin, can probably serve as stimulators of pro-MMP-1 production.(57)

GXXPG sequence peptides can lead to a consequent degradation of collagen and other matrix components. This phenomenon would play an important part in the mechanisms controlling connective tissue remodeling during normal aging and/or pathological processes. (57)

The biosynthesis of proteins, collagen and fibronectin by human skin fibroblasts were evaluated in the presence of agonists and antagonists of the elastin-laminin receptor (ELR). 1 microgram/ml kappa-elastin (EP) in the culture medium increased total proteins and fibronectin biosynthesis. Melibiose, an antagonist of the receptor decreased both total protein and fibronectin biosynthesis at the 10^{th} and 15^{th} passage at a concentration of 5 micrograms/ml (140 micromolar) in the culture medium. Thus the ELR can control the biosynthesis of some constituents of the extracellular matrix and its effect can be modulated by galactosides such as melibiose in an age and passage dependent upregulation. (56)

The 67-kD elastin-laminin receptor (ELR) functions as a lectin and carries the recognition site for elastin peptides (EP) which serve as a ligand. Galactosides such as lactose or meliobiose can modulate the kinetics of ELR-EP binding.(56)

Elastin peptides and Melibiose were evaluated for their effect on the biosynthesis of glycosoaminoglycans(GAG) and hyaluronan on cultures of human skin fibroblasts. Newly synthesized GAGs were excreted into the extracellular medium. Incorporation of the tracer in hyaluronin increased with passage number but its titratable concentration decreased with in vitro aging, suggesting rapid post synthetic degradation. The proportion of chondroiten sulfate4 and 6 and heparin sulfate decreased and that of dermaten sulfate increased with increasing passage number. Both elastin peptide and melibiose increased the incorporation of the tracer in GAGs, but only meliobiose inhibited post-synthetic degradation of hyaluronan, thereby increasing its concentration.(9)

Human lymphocytes have been shown to express the elastin-lamin receptor (ELR). In the presence of elastin peptides (EP) the receptor was shown to increase cell proliferation and increase the synthesis of an elastin-type serine endopeptidase. Elastase and cathepsin G activity increased at increasing concentrations of EP up to 100 micrograms/ml resulting in a dose-dependent increase in cell death. Elastin peptide-induced cell death was inhibited by 1 microgram/ml lactose and melibiose. “The mechanism of cell death appears to be related to the triggering of the release of elastase and free radicals mediated by the elastin-lamin receptor. Antagonists of this receptor, lactose and melibiose, protected the lymphocytes from receptor-mediated cell death. (65).

Collagen Molecules. Within an individual collagen molecule, the three polypeptide strands are linked together by stable intramolecular bonds that originate in the non-helical ends of the molecule. The great strength of collagen fibers, however, originates mainly from the stable intermolecular covalent bonds between adjacent tropocollagen molecules. Stable disulphide bonds between cystine molecules in the triple helix also occur. During the growth and development of animals, covalent cross links increase in number, and collagen fibers from older animals contain progressively more cross linkages. This difference in the age related ratio of cross-linkages is magnified by the rapid synthesis of large amounts of new collagen in young animals. New collagen has fewer cross links so that, if there is a high proportion of new collagen, the mean degree of cross linking may be low, even though all existing molecules are developing new cross links. As the formation of new collagen slows down, the mean degree of cross linking increases. Another complication is that many of the intermolecular cross links in young animals are reducible (the collagen is strong but is fairly soluble). In older animals, nonenzymic glycosylation (the Maillard Reaction) also occurs, forming non-reducible cross links. The rate of collagen turnover is therefore reduces as the animal ages.

### BRIEF SUMMARY OF THE INVENTION

In one embodiment, compositions are provided by the present invention for topical skin treatments to: 1. Attract fibroblasts and monocytes into the area of application (chemotaxis); 2. To stimulate fibroblasts to produce certain Matrix Metalloproteinases (MMPs) which have been shown to be able dissolve cross-linked collagen and elastin fibers; 3. To stimulate the production of elastin-type serine endopeptidase (elastin and cathepsin G); 4. To stimulate the production of collagen and elastin; 5. To stimulate the production of certain Glycosaminoglycans(GAG), Proteoglycans, Hyaluronin and fibronectin; 6. To modulate the above reactions so as to result in a net gain of collagen, elastin and hyaluronin in the treated tissues. These interactions aid in remodeling aged and damaged skin by promoting the catalytic breakdown of highly cross linked, thickened, damaged and otherwise insoluble collagen and elastin, and promoting the synthesis of new collagen, elastin, fibrillin and glcosaminoglycans.

In another embodiment, there is disclosed such composition where the elastin peptide is encapsulated in liposomes or microsponges adapted to aid in delivery of the peptides, or to enhance the stability of the composition. In yet another embodiment, the components of the disclosed compositions are formulated in an instrument adapted to deliver the components via ionotophoresis.

In another embodiment, there is disclosed compositions of the invention formulated specifically for skin application in conjunction with the practice of needling the skin. The needling technique is usually but not always performed with needled rollers. The needle penetrates through the epidermis and does not remove it. The needle channel which penetrates the epidermis allows a path for the herein described invention to penetrate to the dermis. Concentrations of elastin peptide and galactosugars can therefore be formulated on the basis of concentrations with desired effects in cell culture experiments.

Additional embodiments of this invention are directed to the above compositions that further include an inert carrier or diluent, a sunscreen agent, a skin protectant, an emollient, a humectant, an excipient, a textural modifier, an emulsifying agent, a preserving agent, a thickening agent, or a mixture thereof. These compositions may be in the form of a solution, cream, gel, fluid cream, serum, lotion or oil. Pharmaceutical and cosmetic preparations for skin, made from these compositions, are also disclosed.

The present invention also discloses a method for treating skin by contacting the skin with an effective amount of a disclosed inventive composition or preparation. The effect of such treatment include conditioning and smoothing the skin, reducing signs of aging and photodamage, and reducing hyperpigmentation and wrinkling of the skin.

### DETAILED DESCRIPTION OF THE INVENTION

As noted above, in one embodiment, disclosed is a composition formed by combining an elastin peptide of the general formula (GXXPG)n and in particular the sequences of (VGVAPG)n, and/or (GVAPGV)n, and/or (VAPGVG)n and/or (APGVGV)n and or (PGVGVA)n and/or (GVGVAP)n and various carrier substances. A galactosugar may be added to modulate the effects of the elastin peptide on the production of certain metalloproteinases.

wherin

A is a peptide-forming residue of L-alanine;
G is a peptide-forming residue of glycine
P is a peptide-forming residue of L-proline
V is a peptide-forming residue of L-valine
X is a peptide-forming residue of a single unspecified amino acid
   Peptide representations in this application conform to the standard practice of writing the NH₂-terminal amino acid residue at the left of the formula and the CO₂ H-terminal amino acid residue at the right.
   When an elastin polypeptide(EP) is present, the compound is chemotactic and stimulatory regardless of the value of n. However, higher values of n are less soluble and are less likely to penetrate the epidermis. Preferred are values of n are from 1 to 10. Synthesis of these elastin peptides is easily accomplished by a protein chemist and are commercially available in pharmaceutical grade.

In certain specific embodiments the composition of the present invention comprises at least one elastin peptide that is (VGVAPG)n, and/or (GVAPGV)n.

Galactosugars are commercially available in pharmaceutical grade.

The disclosed composition provides topical formulations effective for the treatment and prevention of photodamaged skin, the appearance of fine lines and wrinkles, hyperpigmentation, age spots, and aged skin. The composition aids in the removal of thickened and damaged skin collagen and elastin and promotes the formation of new collagen and elastin. The disclosed composition also increases the amount of glycosaminoglycans in the skin thus increasing moisture in the skin.

The composition intended for topical administration may be in the form of a liquid, solid or semi-solid such a as a lotion, serum, cream, salve, ointment, suspension, liposome or paste and may be compounded with conventional nontoxic carriers such as, for example, aloe vera gel, squalene, glycerol stearate, polyethelene glycol, cetyl alcohol, stearic acid, propylene glycol, dimethicon, dimethiconol, and acrylates amongst others. In addition it may contain other medicinal agents.

In addition to the active ingredients described above, the disclosed compositions and preparations may contain inert carriers and/or diluents.

Compositions will consist of conventional nontoxic carriers buffered to physiologic pH. To this base Elastin Peptide(s) as described above are added. The concentration will vary depending upon the vehicle of administration and the condition of the underlying substrate skin and is compounded to achieve an intended concentration of 5-200 micrograms/ml at the dermal-epidermal junction. To this composition a galactoside such as but not limited to melibiose or lactose may be added at a variable amount.

As different individuals have skin with variable properties that can effect the absorption of the therapeutic ingredients differing compositions will be formulated for the various skin substrates. Conditions that may affect absorption are: 1. Age of the individual; 2 use of skin preparations i.e. exfolliants such as glycolic acid or salicylic acid; 3. genetic difference in skin texture; 4. nutritional state of the individual; 5. amount of cross-linked fiber deposition; 6. use of mechanical abrasives and scrubs; 7. exposure to defatting and/or irritant chemicals and other skin irritants such as UV light.

Determining actual amounts of elastin peptide, galactosides and carriers necessary to achieve a desired effect in a particular skin type or condition will be through standard empirical methods well known in the art. The compositions described herein stimulate a spectrum of healing processes.

Specific embodiments of the invention have been described herein for purposes of illustration. Modifications may be made without deviating from the scope of the invention. Accordingly, the invention is not limited except as by the appended claims.

1. A topical composition containing an elastin peptide of the general formula (GXXPG)n and in particular the sequences of (VGVAPG)n and/or (GVAPGV)n, and/or (VAPGVG)n and/or (APGVGV)n and or (PGVGVA)n and/or (GVGVAP)n and a galactoside.
wherin
a. A is a peptide-forming residue of L-alanine;
b. G is a peptide-forming residue of glycine
c. P is a peptide-forming residue of L-proline
d. V is a peptide-forming residue of L-valine
e. X is a peptide-forming residue of a single unspecified amino acid Peptide representations in this application conform to the standard practice of writing the NH₂-terminal amino acid residue at the left of the formula and the CO₂H-terminal amino acid residue at the right.

2. A method of modulating the effect of the elastin peptide on the production of Matrix Metallolproteinases and other collagenolytic and elastinolytic enzymes by the addition of galactosugar-bearing moieties such as but not limited to Lactose or Melibiose.

3. A method for cosmetically treating skin by direct application of an effective amount of the composition of claim 1 and claim 2 to an area of skin in need thereof.

4. The method of claim 3 wherein the cosmetic treatment of skin reduces wrinkles in the skin, reduces evidence of photodamage to the skin, increases water content in the skin, smoothes the skin, reduces skin discoloration, reduces signs of aging in the skin.

5. A method of claim 3 wherein the composition of claim 1 act to attract fibroblasts into the dermis in the area of application. (chemotaxis)

6. A method of claim 3 wherein the composition of claim 1 stimulates the production of enzymes capable of dissolving otherwise insoluble and cross-linked collagen and elastin fibrils.

7. A method of claim 3 wherein the composition of claim 1 acts to stimulate the production of proteins, glycoproteins, glycosaminoglycans and hyaluronin by skin fibroblasts.

8. A method of claim 3 wherein the composition of claim 1 stimulates the production of tropoelastin and elastin by skin fibroblasts

9. A method of claim 3 wherein the composition of claim 1 stimulates the production of tropocollagen and collagen.

10. A method of claim 3 wherein the composition of claim 1 and claim 2 modulates and reduces the production of certain matrix metalloproteinases, elastin-type serine endopeptidase, elastase and cathepsin G thus allowing for the net increase of elastin, collagen, glycosaminoglycans and hyaluronin.
